(19) United States
(12) Patent Application Publication
(10) Pub. No.: US 2007/0160492 A1
(43) Pub. Date: Jul. 12, 2007

(54) Electronic room freshener and method for releasing fragrances

(76) Inventor: Donald Spector, New York, NY (US)

Correspondence Address: Glen M. Diehl;NORTON & DIEHL LLC, Suite 110, 77 Brant Ave., Clark, NJ, 07066, US

(21) Appl. No.: 11/329,653

(22) Filed: Jan. 11, 2006

(51) Int. Cl. A61L 9/03; A61L 9/02

(52) U.S. Cl. 422/5; 422/125

(57) ABSTRACT

Systems and methods for dispensing fragrances are disclosed. A system, in accordance with one aspect of the present invention includes a heat source that emits a varying amount of heat over time, a pad located near the heat source such that the pad receives heat from the heat source, a container for the one or more fragrances, and a dispenser located between the container and the pad such that the one or more fragrances can be dispensed onto the pad.

### CROSS-REFERENCE TO RELATED APPLICATIONS

This application claims the benefit of U.S. Provisional Application No. ______, filed Dec. 21, 2005, which is incorporated herein by reference.

### BACKGROUND

This invention relates to the field of electronic air fresheners. Currently, a major product in this industry is the Aroma Disc Player. This product uses fragrance records which are embedded with fragrance. They are inserted into a player that gives off a heat beneath the record and vaporizes the fragrance oil. This product remains one of the finest fragrance release systems available.

A drawback of this system is that The Aroma Disc is meant to be played for a few hours and then turned off. This design does not satisfy an increasing consumer demand for longer term players that can be turned on and left on.

Products like fragrance nightlights or the SC Johnson Plug Ins® lack the ability to provide varying amounts of fragrance. Without these changing levels of fragrance, a person becomes accustomed to a fragrance level and cannot appreciate it over a longer period of time.

Accordingly, there is a need in the industry for fragrance dispensers and methods of dispensing fragrances that provide variable amounts of fragrance for a long time without a user of the product becoming desensitized to the scent.

### SUMMARY OF INVENTION

The present invention provides systems and methods for dispensing fragrances. In accordance with one aspect of the present invention, a system for dispensing one or more fragrances includes a heat source that emits a varying amount of heat over time, a pad located near the heat source such that the pad receives heat from the heat source, a container for the one or more fragrances, and a dispenser located between the container and the pad such that the one or more fragrances can be dispensed onto the pad.

In accordance with another aspect of the present invention, a controller that electronically controls the amount of heat emitted by the heat source is provided. A valve that controls the amount of the one or more fragrances dispensed by the dispenser can also be provided. The position of the valve can be electronically controlled by a controller to determine the amount of fragrance released onto the pad.

The system can also include a first roller at one end of the pad and a second roller at another end of the pad to allow new areas of the pad to be exposed. A handle can be provided on one of the rollers to allow them to be manually turned, or alternatively, the controller can electronically control the rollers to periodically turn the rollers to cause new areas of the pad to be exposed.

A method in accordance with one aspect of the present invention includes the steps of dispensing a fragrance onto a pad and varying an amount of heat emitted by a heat source over time, the heat source being located near the pad so that the pad receives a varying amount of heat. This causes a varying amount of fragrance to be emitted from the pad over time.

In yet another aspect, the system of the present invention is housed in a consumer electronic device, such as a tape player, a CD player, a computer, a DVD player, or a video tape player.

### BRIEF DESCRIPTION OF THE FIGURES

FIG. 1 illustrates one embodiment of the scent dispensing system of the present invention.

FIGS. 2 to 8 show exemplary time-varying heater control as provided by various aspects of the present invention.

FIG. 9 illustrates another embodiment of the scent dispensing system of the present invention.

### DETAILED DESCRIPTION

The present invention uses a heat source. The source varies in intensity so that it does not maintain a single level of heat. This is important in creating fragrance bursts that can break up the fragrance level in a room. Above this heat source is a pad that is made of an absorbent material that also will not burn. The substances, such as carborundum, can be on a roll that is either mechanically wound (like a towel dispenser) or automatically turned. The automatic turning can be performed every day or every second day. Since there is a small surface area over which the fragrance is vaporized, such a roll can last an extremely long period of time. Alternatively, a self cleaning system can be provided. The self cleaning system can be made of a gauze type ceramic or permanently absorbent material that has to be changed every month when it clogs. In this case, a mechanical roller is not needed.

In accordance with one aspect of the present invention, to provided fragrance oils to the substrate on a continuous basis, a liquid chamber or cartridge can be combined with tubing and drip technology (similar to an IV drip). This simple system will enable the fragrance oil to be applied to the substrate on a continuous basis. Just like an IV tube, there is preferably a valve that will enable the user to control the fragrance level by regulating the drip level.

The combination of the cartridge, the drip tube and the changing heat source enables the system to function as a long term fragrance delivery system.

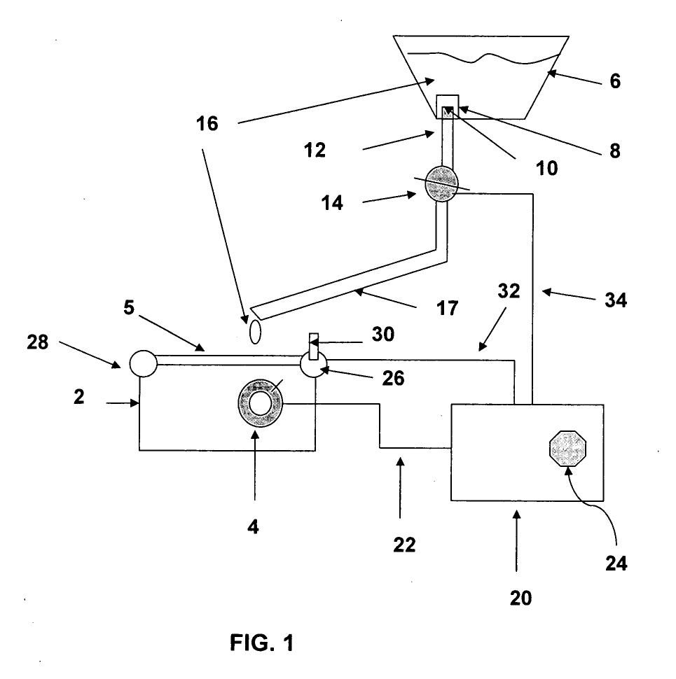

FIG. 1 illustrates an embodiment of the system of the present invention. The system comprises a heat source 2, which may optionally include a heat regulator 4 allowing the amount of heat emitted by a pad 5. The pad 5 is located near the heat source 2 so that the pad 5 is heated by the heat source 2. In accordance with one embodiment of the present invention, the pad 5 may be in contact with the heat source 2.

The pad 5 is preferably constructed from an absorbent material that will not burn or that is non-flammable. The pad 5 may be made from any suitable material, including any ceramic materials, aluminum oxide, silicon carbide, and/or carborundum available from Saint-Gobain.

In operation, when the fragrance 16 hits the pad 5 that has been heated by the heat source 2, the fragrance is vaporized. The vaporized fragrance 16 is then emitted into the air from the pad 5.

The system further includes a container, housing or cartridge 6, containing a fragrance 16. The cartridge may also contain one or more fragrances. The cartridge 6 includes a port 8. The port 8 receives one end 10 of a tube 12. The port 8 and the end 10 can be threaded. Alternatively, the port 8 and the end 10 can be fit together with a press fit. When the cartridge 6 and tube 12 are properly connected, they preferably form a liquid tight seal. There are multiple ways of connecting the cartridge 6 and the tube 12.

In accordance with one embodiment of the present invention, a valve 14 is connected to the tube 12. The valve 14 is used to regulate the amount of fragrance 16 which is dispensed from a second tube 17 onto the pad 5. The valve 14 can be similar to the valves used, for example, in IV drips.

A controller 20 is provided. The controller 20 can be a microprocessor based circuit, a hardwired circuit or other suitable electronic controlling devices. The controller 20 has a connection via wire 22 to the heat regulator 4. The controller 20 provides a time-varying output electrical signal to the heat regulator 4 to control the amount of heat emitted by the heat source 2. The controller 20 can, for example, provide a square wave to the heat regulator 4 to turn the heat source 2 on and off on a periodic basis. For example, the controller 20 can cause the heat source 2 to be turned on at a certain time every day. The heat source 2 can then be cycled on and off as necessary to prevent people from becoming accustomed to the scent.

The controller 20 can control the heat regulator 4 in a number of well known ways. For example, the output of the controller 20 can be any number of periodically varying signals, for example, as a square wave, a sine wave, a saw tooth wave. The frequency or duty cycle of the output can control the amount of heat emitted. For example, a higher frequency signal would cause more heat to be emitted than a lower frequency signal. By outputting a voltage of 0 volts, the controller 20 can turn the fragrance delivery system off.

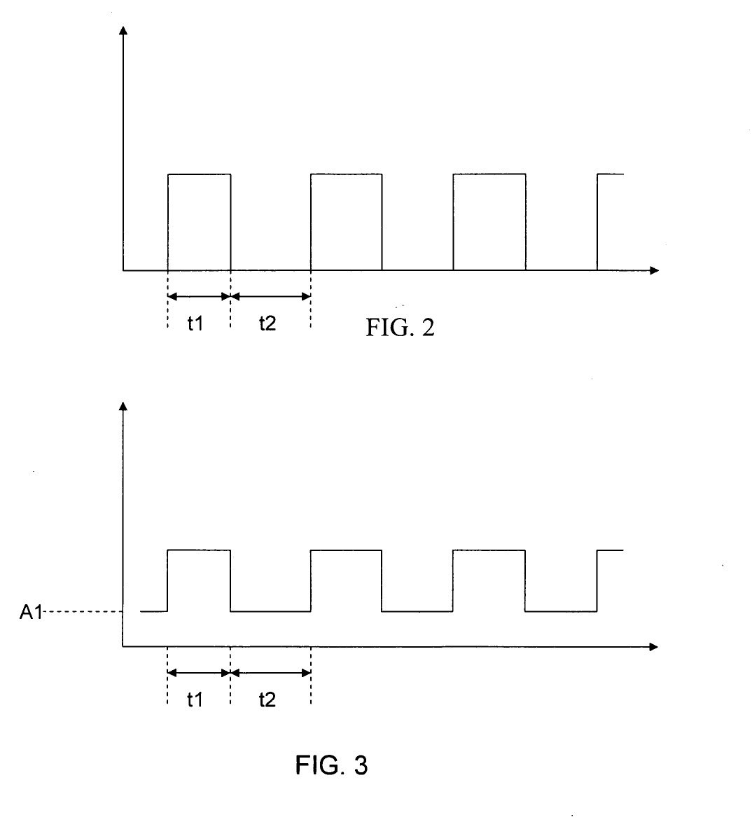

FIGS. 2 to 8 provide exemplary graphs of the output signal versus time that may be provided by the controller 20 on the wire 22 to control the heat regulator 4, and hence control the temperature and duration of heat provided by the heat source 2. The amplitude of the example signals shown in FIGS. 3 to 8 may represent, for example, the voltage present on wire 22, the current passing through wire 22, or, in the event that the heat regulator 4 accepts a digital control input, the digital value transmitted via the wire (or wires) 22. Regardless of the method used to pass signal information to the heat regulator 4, for purposes of the disclosure, higher amplitudes in the graphs indicate that more power should be provided by the heat regulator 4 to the heat source 2, and hence indicate higher temperatures provided by the heat source 2. The horizontal axis represents time, but is not necessarily to scale, as will become clear from the following.

With reference to FIG. 2, one possible pattern for the control signal on wire 22 is a simple square wave having zero bias. Hence, during period t1, the heat source 2 is fully on, so that fragrance is distributed by the pad 5. During period t2, the heat source 2 is turned off, and so little or no fragrance is distributed. This cycling alternates the distribution of fragrance, so that variations of the level of fragrance are provided, and hence the user does not become accustomed to a constant background fragrance. The period t1 need not be of the same duration at the period t2. Duration t1 may, for example, be on the order of a few minutes to an hour or more. Duration t2 is preferably not so long that fragrance is entirely lost from the environment. For example, it may be desirable that t2 not exceed 90 minutes in length. Also, it may be preferable that t1 exceeds t2 in duration, though this is certainly not a requirement. Further, the amplitude of each cycle may vary as well.

The waveform depicted in FIG. 3 is substantially the same as that of FIG. 2, except that the controller 20 provides a signal with a non-zero bias, as indicated by amplitude A1. Hence, the waveform in FIG. 3 oscillates between a full-on state and a partially on state. The heat source 2 is thus never fully turned off, but instead delivers varying doses of high concentrations of fragrance during period t1, and relatively lower doses of fragrance during period t2. For example, the waveform of FIG. 3 may cause heat source 2 to deliver four times as much fragrance during period t1 than period t2.

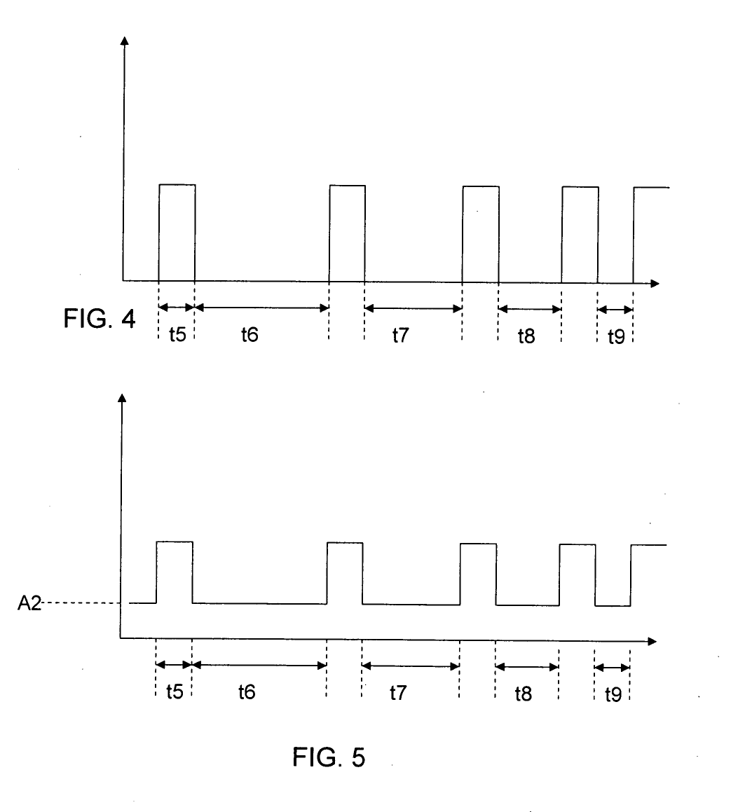

Under certain conditions it may be desirable to vary the frequency with which fragrance is delivered, i.e., change the duty cycle of the waveform, and this is exhibited in FIG. 4. FIG. 4 shows a waveform provided by the controller 20 in which the duty cycle steadily increases. During “on” periods, as indicated by period t5, the pad 5 releases fragrance. In “off” period t6, the heat source 2 is not active and so little or no fragrance is delivered. These periods of “off” and “on” occur repetitively, so that fragrance is distributed in a non-constant manner, and therefore the background perception of fragrance is continuously changing. However, as shown in FIG. 2C, the “off” periods may become successively shorter. So, “off” period t7 is shorter than immediately proceeding “off” period t6. Similarly, period t8 is shorter than period t7, and period t9 is shorter than period t8.

The waveform depicted in FIG. 5 is substantially the same as that depicted in FIG. 5, but has a non-zero bias, as indicated by amplitude A2. Hence, the heat source 2 never fully shuts off. Fragrance is instead delivered in relatively high bursts followed by relatively low bursts.

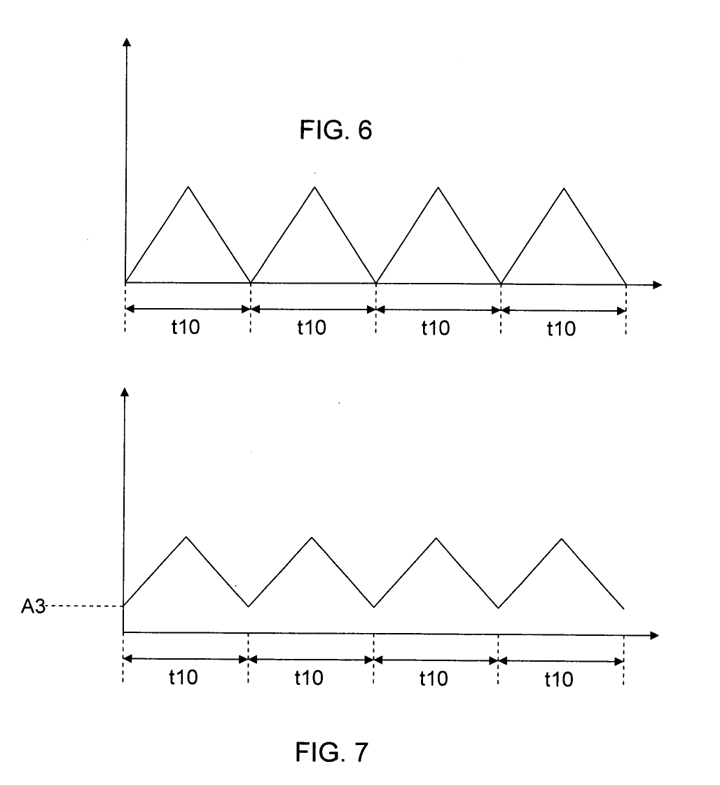

Of course, square waveforms need not be exclusively used. As shown in FIG. 6, a saw-toothed pattern for the control signal 22 may be employed. In this case, over a repetitive period t10, the heat source 2, and hence the delivery dosage of fragrance, slowly ramps up to a maximum, and then ramps back down to zero. The period t10 may be as short as a few minutes, such as ten minutes, to as long as a few hours, such as three hours. FIG. 7 is substantially similar to FIG. 6, but provides a non-zero bias A3 so that the heat source 2 never fully turns off.

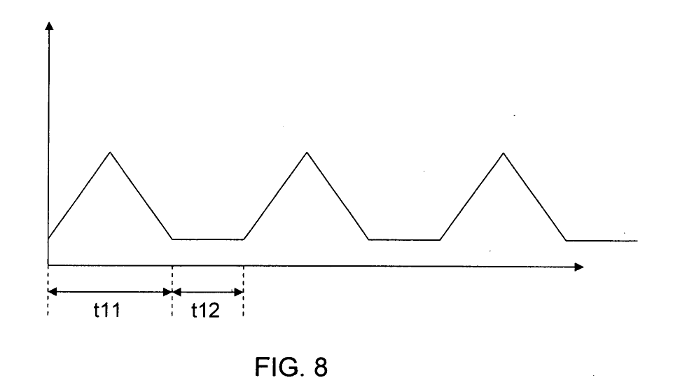

As shown in FIG. 8, it is also possible to introduce delays between the ramping periods of increasing and decreasing fragrance delivery doses. For example, over period t11, the heat source 2 ramps from a minimum heating value up to a maximum value, and then back down to the minimum value. Then, over the course of period t12, the heat source 2 is kept at the minimum value. The cycle then repeats. Of course, FIG. 8 presents the situation in which a non-zero bias is present. It should be clear, however, that a zero-bias waveform may also be used, or even a bias that changes as a function of time. Also, it should be clear that other functions other than square waves and saw-toothed waves may be used. For example, the well-known ramp function may be used, or a sinusoidal function may be employed.

With reference to FIG. 1, the heat regulator 4 may alternatively be manually controlled via a control knob to allow the heat emitted by the heat source 2 to be varied. Or, the controller 20 may be provided one or more user controls 24 that enable the user to change the shapes, biases and periods of the waveforms provided by the controller 20 to the heat regulator 4. For example, the user control 24 may enable the user to change the duty cycle of the waveform, the amplitude of the waveform, the duration of the “on” periods, the bias, etc.

Thus, the present invention, by varying the amount of heat emitted by the heat source 2, controls the amount of fragrance 16 emitted by the pad 5 during operation. By varying the amount of fragrance 16 emitted, the fragrance delivery system is more effective as people do not become accustomed to the fragrance.

A first roller 26 and a second roller 28 can be provided. The pad 5 is attached to the first roller 26 and to the second roller 28 so that when the rollers 26 and 28 turn, new areas of the pad 5 are exposed. If the rollers 26 and 28 are not provided, then a new pad 5 should be periodically installed.

A handle 30 can be connected to one of the rollers 26 or 28. The handle 30 allows a user to manually turn the rollers 26 and 28 so that new areas of the pad 5 are exposed.

Alternatively, the controller 20 can be connected to the roller 26 via a wire 32. The controller 20 can cause the roller 26 to be rotated a fixed amount on a periodic basis so as to provide a fresh area of the pad 5 for the fragrance 16.

The controller 20 can also be electrically connected to the valve 14 via a wire 34. The controller can control the operation of the valve 14 to control the amount of fragrance 16 dispensed onto the pad 5. This control can once again be effectuated on a periodic basis and can be cycled to help control the amount of fragrance emitted from the pad 5.

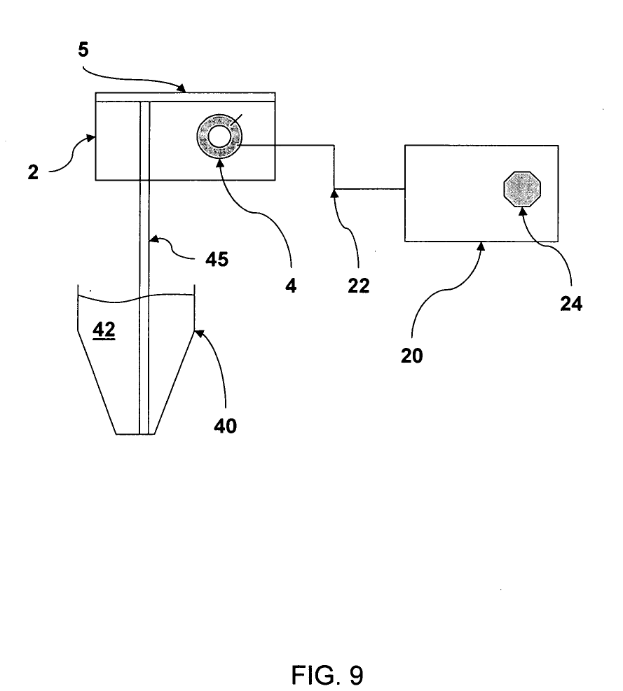

An alternative embodiment is presented in FIG. 9, in which items that are substantially similar are given the same reference numbers as those in FIG. 1. Rather than using the drip system of the embodiment in FIG. 1, a capillary or wick system is instead employed, which includes liquid fragrance 42 held by a reservoir 40, and a wick 45. One end of the wick 45 is disposed within the liquid fragrance 42, while the other end 45 is connected to the pad 5. As the pad 5 dries out, capillary action draws the liquid fragrance 42 through the wick 45 to wet the pad 5. Although depicted as separate items, it should be noted that the wick 45 and the pad 5 may be made form the same piece of material; that is, the wick 45 may simply be an extension of the pad 5.

While there have been shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the intention, therefore, to be limited only as indicated by the scope of the claims appended hereto.

1. A system for dispensing one or more fragrances, comprising:
a controller capable of providing a time-varying output;
a heat source that emits a varying amount of heat over time according to the time-varying output;
a pad located near the heat source such that the pad receives heat from the heat source;
a container for the one or more fragrances; and,
a dispenser located between the container and the pad such that the one or more fragrances can be dripped onto the pad.

2. The system of claim 1, further comprising a controller that controls the amount of heat emitted by the heat source.

3. The system of claim 1, further comprising a valve that controls the amount of the one or more fragrances dispensed by the dispenser.

4. The system of claim 3, further comprising a controller that controls the valve.

5. The system of claim 1, further comprising a first roller at one end of the pad and a second roller at another end of the pad.

6. The system of claim 5, further comprising a handle on the first roller, wherein the handle can be turned to expose new areas of the pad

7. The system of claim 5, further comprising a controller connected to the first roller to control the first roller.

8. The system of claim 2, further comprising a valve that controls the amount of the one or more fragrances dispensed by the dispenser and wherein the controller controls the valve.

9. The system of claim 2, further comprising a first roller at one end of the pad and a second roller at another end of the pad and wherein the controller is connected to the first roller to control the first roller.

10. The system of claim 1, wherein the pad is a non-flammable, absorbent material.

11. A method for dispensing one or more fragrances, comprising:
dripping a fragrance onto a pad;
varying an amount of heat emitted by a heat source over time, the heat source being located near the pad so that the pad receives a varying amount of heat;
whereby a varying amount of fragrance is emitted from the pad over time.

12. The method of claim 11, further controlling the amount of heat emitted by the heat source with a controller.

13. The method of claim 11, further comprising controlling the amount of the fragrance dispensed onto the pad with a valve.

14. The method of claim 13, further comprising controlling the operation of the valve with a controller.

15. The method of claim 11, further comprising rotating the pad on a first roller at one end of the pad and a second roller at another end of the pad to expose new areas of the pad.

16. The method of claim 15, further comprising manually rotating the pad.

17. The method of claim 15, further comprising rotating the pad electronically via a controller.

18. The method of claim 12, further comprising controlling the amount of the one or more fragrances dispensed with a valve.

19. The method of claim 10, wherein the pad is a non-flammable, absorbent material.

20. A system for dispensing one or more fragrances, comprising:
a controller capable of providing a time-varying output;
a heat source that emits a varying amount of heat over time according to the time-varying output;
a pad located near the heat source such that the pad receives heat from the heat source;
a container for the one or more fragrances; and,
a tube connected between the container and the pad such that the one or more fragrances is dispensed to the pad via a capillary action.
